(19) United States
(12) Patent Application Publication
(10) Pub. No.: US 2021/0256099 A1
(43) Pub. Date: Aug. 19, 2021

(54) INFORMATION PROCESSING METHOD

(71) Applicant: NEC Corporation, Minato-ku, Tokyo (JP)

(72) Inventors: Tomohiro MIWA, Tokyo (JP); Joji TANAKA, Tokyo (JP); Yoshikazu ARAI, Tokyo (JP); Keiji KANAMEDA, Tokyo (JP); Tsuyoshi NAKAMURA, Tokyo (JP); Yuki KOBAYASHI, Tokyo (JP)

(73) Assignee: NEC Corporation, Minato-ku, Tokyo (JP)

(21) Appl. No.: 17/271,270

(22) PCT Filed: Dec. 3, 2019
(86) PCT No.: PCT/JP2019/047246

§ 371 Date: Feb. 25, 2021

(30) Foreign Application Priority Data
Jan. 18, 2019 (JP) 2019-006746

(51) Int. Cl. G06F 21/31; G06K 9/00; G06K 9/62

(52) U.S. Cl. CPC G06F 21/31; G16H 40/67; G06K 9/6201; G06K 9/00362

(57) ABSTRACT

An information processing method includes acquiring image information imaged in such a manner that identification information for identifying a user and a scene related to the user are associated with each other (step S11), and performing a process of associating the identification information registered in advance with the image information including the scene related to a user who is the same as the user identified by the identification information (step S12).

### TECHNICAL FIELD

The present invention relates to an information processing method, a program, an information processing device, and an information processing system, for managing images.

### BACKGROUND ART

Recently, it is easy to capture images such as videos using a mobile terminal such as a smartphone or a tablet terminal. Along with it, situations of managing captured images are increasing. In particular, an operator who operates a management system for managing images often manages images of a plurality of users. As an example, as described in Patent Literature 1, there is a case where a salesperson of a shop registers video data, stored in a mobile phone of a user, with a management server. As another example, there is a case where a nurse or a caregiver manages the health condition of patients and those who need nursing care in the medical and nursing-care filed, by means of text data and motion videos. As still another example, there is a case where an employee of a non-life insurance company manages images capturing the conditions of the site and registration information in association with each other, when a user having a vehicle registered with the management system caused an accident.

When an operator who operates a management system registers images related to a plurality of users to be managed, the operator performs operation as shown in FIG. 1, for example. First, the operator registers user information that is information about each user with the management system, from an information processing device such as a personal computer (S101). Thereafter, the operator captures a video of each user by a mobile terminal such as a smartphone or a tablet terminal (S102), associates the video with the user information registered in advance, and uploads to the management system (S103). In that case, the operator first logs in to the management system from the information processing device and registers user information, and then logs in to the management system again from a video capture application (app) on the mobile terminal, and selects a corresponding user. Thereby, the video and the user are managed in association with each other.

### CITATION LIST

### Patent Literature

Patent Literature 1: JP 3149786 U

### SUMMARY

However, a series of works for image registration by the operator as described above involves a problem as described below. First, when the operator selects a user to be associated with a captured image, a burden of taking caution to prevent erroneous selection of a user is imposed on the operator. In particular, as the number of selectable users increases, a larger burden is imposed on the operator. Moreover, after the operator logs in to the management system from an information processing device to register user information, the operator needs to perform a login process on the management system from each mobile terminal that captured the image, to register the image. Therefore, a burden of an operation of inputting a password a number of times may be caused. In particular, for those who are not familiar with an information processing device, when a user interface is different because the device is different, a further burden may be imposed.

Therefore, an object of the present invention is to provide an information processing method, a program, an information processing device, and an information processing system, capable of solving the aforementioned problem, that is, a problem that a burden is imposed on an operator who manages images.

An information processing method that is one aspect of the present invention includes

acquiring image information imaged in such a manner that identification information for identifying a user and a scene related to the user are associated with each other; and

based on the image information, performing a process of associating the identification information registered in advance with the image information including the scene related to a user who is same as the user identified by the identification information.

Further, a program that is one aspect of the present invention is a program for causing an information processing apparatus to realize:

a process of acquiring image information imaged in such a manner that identification information for identifying a user and a scene related to the user are associated with each other, and based on the image information, performing a process of associating the identification information registered in advance with the image information including the scene related to a user who is the same as the user identified by the identification information.

Further, an information processing device that is one aspect of the present invention includes

an acquisition unit that acquires image information imaged in such a manner that identification information for identifying a user and a scene related to the user are associated with each other; and

a processing unit that performs a process of associating the identification information registered in advance with the image information including the scene related to a user who is same as the user identified by the identification information.

Further, an information processing system that is one aspect of the present invention includes

a first device that outputs identification information so as to be imagable, based on the identification information for identifying the user registered in advance;

a second device that images image information in which the identification information output to be imagable and a scene related to the user identified by the identification information are associated with each other; and

a third device that performs, based on the image information, a process of associating the identification information registered in advance with the image information including the scene related to a user who is same as the user identified by the identification information.

Since the present invention is configured as described above, it is possible to reduce the burden imposed on an operator who manages images.

### BRIEF DESCRIPTION OF DRAWINGS

FIG. 1 is an illustration for explaining a state of registering images related to a plurality of users.

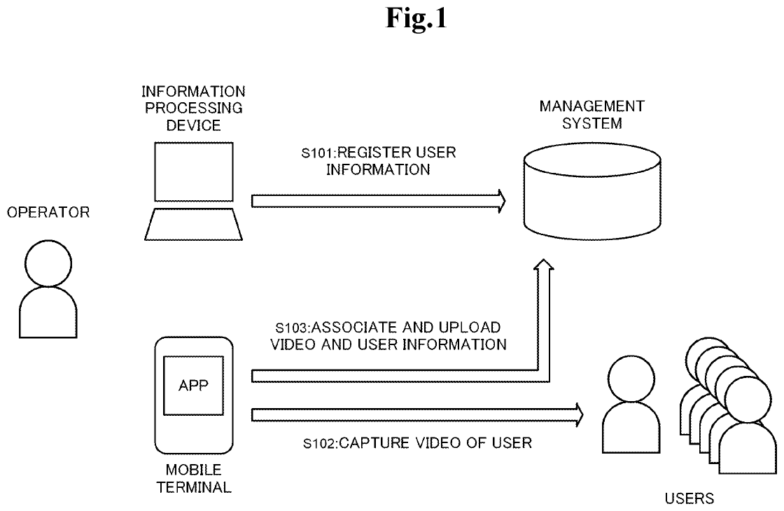

FIG. 2 is an illustration for explaining a scene of using an information processing system for registering images according to a first exemplary embodiment of the present invention.

FIG. 3 is a block diagram illustrating the overall configuration of the information processing system according to the first exemplary embodiment of the present invention.

FIG. 4 is an illustration for explaining an operation of the information processing system disclosed in FIG. 3.

FIG. 5 is a flowchart illustrating an operation of the information processing system disclosed in FIG. 3.

FIG. 6 illustrates a part of the operation of the information processing system disclosed in FIG. 3.

FIG. 7 illustrates an exemplary code generated by the operation illustrated in FIG. 6.

FIG. 8 illustrates a part of the operation of the information processing system disclosed in FIG. 3.

FIG. 9 illustrates a modification of the configuration and the operation of the information processing system disclosed in FIG. 3.

FIG. 10 illustrates a modification of the configuration and the operation of the information processing system disclosed in FIG. 3.

FIG. 11 illustrates a modification of the configuration and the operation of the information processing system disclosed in FIG. 3.

FIG. 12 illustrates a modification of the configuration and the operation of the information processing system disclosed in FIG. 3.

FIG. 13 is a flowchart illustrating an information processing method according to a second exemplary embodiment of the present invention.

FIG. 14 is a block diagram illustrating a configuration of an information processing device according to the second exemplary embodiment of the present invention.

FIG. 15 is a block diagram illustrating a configuration of an information processing system according to the second exemplary embodiment of the present invention.

### EXEMPLARY EMBODIMENTS

### First Exemplary Embodiment

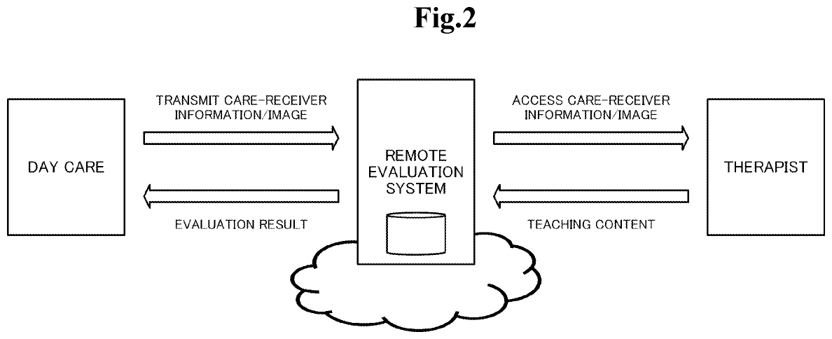

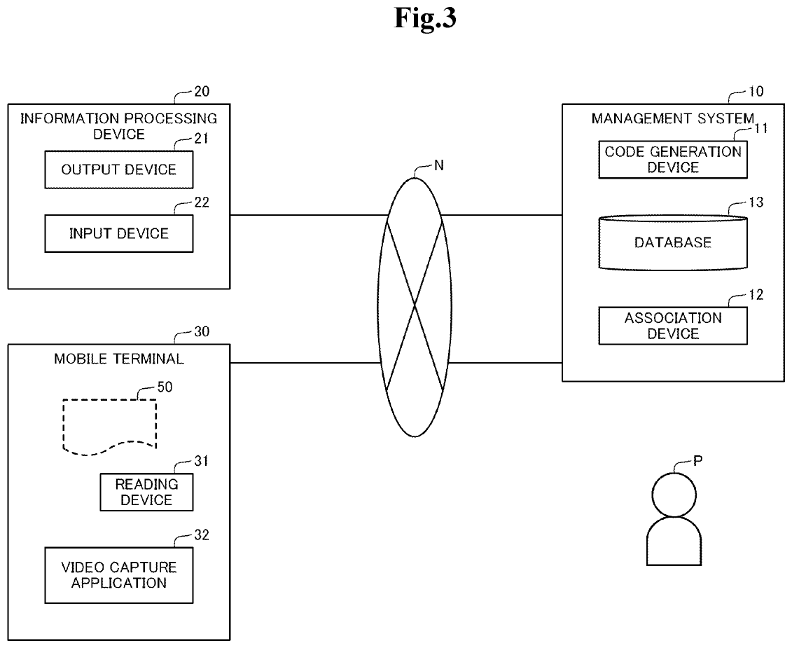

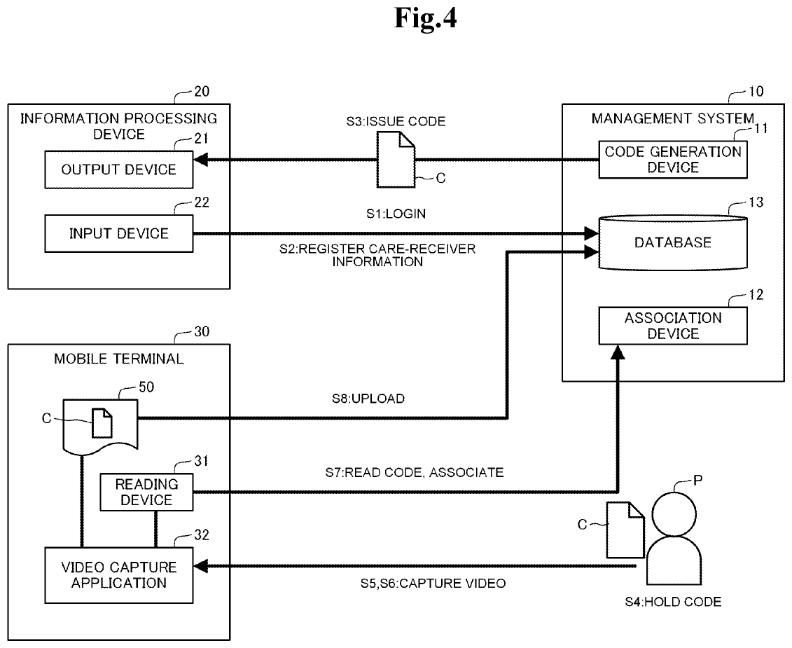

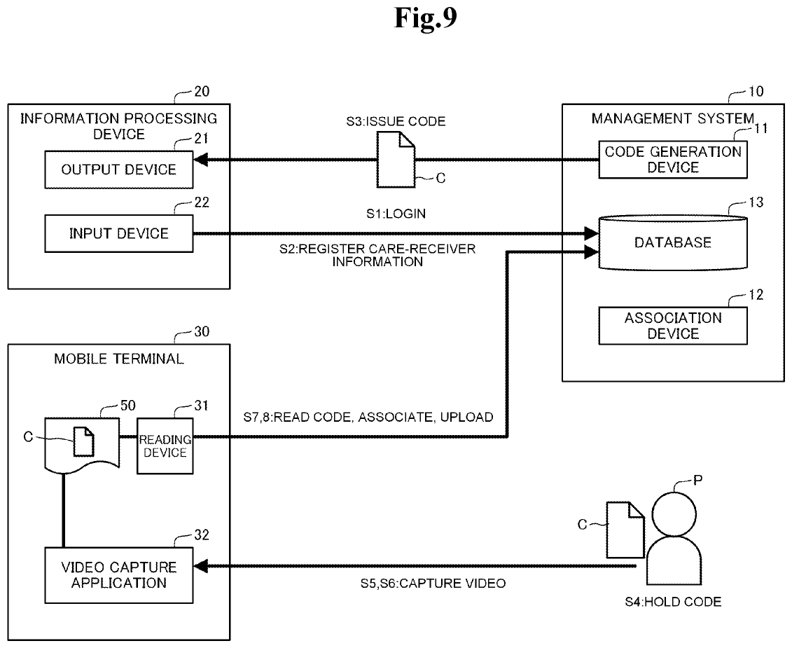

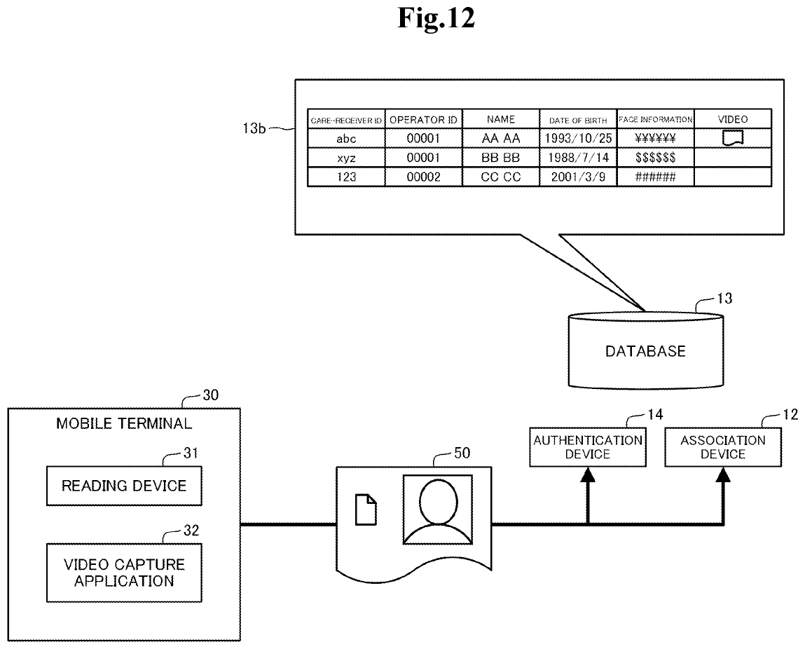

A first exemplary embodiment of the present invention will be described with reference to FIGS. 2 to 12. FIGS. 2 and 3 are illustrations for explaining a configuration of an information processing system. FIGS. 4 to 8 are illustrations for explaining an operation of the information processing system. FIGS. 9 and 12 are illustrations for explaining modifications of the information processing system.

The information processing system of the present invention is for registering images such as videos in association with respective users. As an example, in the present embodiment, description will be given on the case of managing the health condition of patients or those who need nursing care in the medical and nursing-care filed, by means of motion videos. That is, description will be given on the case where an operator who operates the system captures an image such as a video of a care-receiver such as a patient or a person who needs nursing care, and registers the video in association with information of the care-receiver whose video has been captured.

Specifically, as illustrated in FIG. 2, first, in an elderly day care provider, an operator who is a nurse or a caregiver registers information of a care-receiver such as a patient or a person who needs nursing care, and login information of the operator, with the remote evaluation system. Then, the operator uses a mobile terminal such as a smartphone to capture a motion video of a care-receiver such as a patient or a person who needs nursing care, and upload the motion video to a remote evaluation system. At that time, the operator logs in to the remote evaluation system, and registers the motion video in association with information of the corresponding care-receiver. Thereby, in the remote evaluation system, an image of the corresponding care-receiver is registered for each care-receiver.

As described above, since an image of the corresponding care-receiver is registered for each care-receiver, therapists such as a doctor, a physical therapist, and an occupational therapist at remote places can access the image of the care-receiver. Then, based on the accessed image, each therapist creates contents of teaching such as functional training for each care-receiver, and registers them with the remote evaluation system. Thereby, an operator in the elderly day care provider can provide appropriate functional training according to the registered contents of teaching.

Hereinafter, description will be given on an exemplary configuration of an information processing system to be used in the scene as described above. Note that a management system 10, described below, corresponds to the remote evaluation system illustrated in FIG. 2, and an information processing device 20 and a mobile terminal 30 correspond to devices to be used in an elderly day care provider.

As illustrated in FIG. 3, the information processing system of the present embodiment includes the management system 10, the information processing device 20, and the mobile terminal 30, connected over a network N. The management system 10 is a device for registering and managing an image related to a care-receiver P, and the information processing device 20 and the mobile terminal 30 are devices operated by an operator (not shown) who performs an operation for registering images. Hereinafter, configuration and operation of each device will be described in detail.

First, the information processing device 20 (first device) is an information processing device such as a personal computer to be operated by an operator. The information processing device 20 includes output devices 21 such as a display and a printer, and input devices 22 such as a mouse and a keyboard. Note that the functions of the information processing device 20 described below are implemented by a program executed by an arithmetic unit of the information processing device 20.

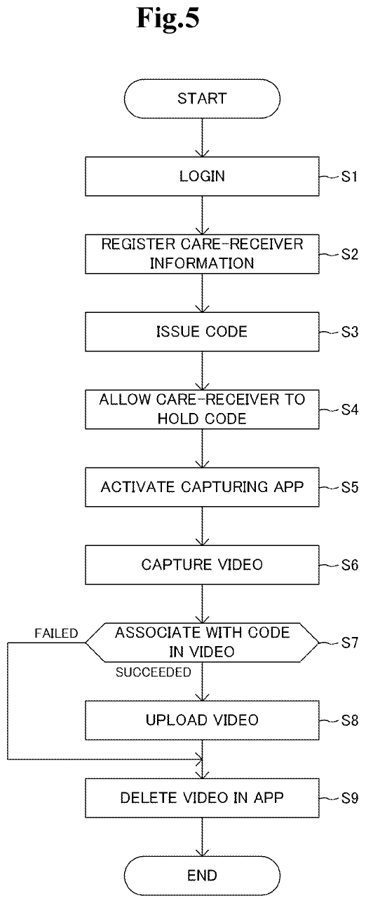

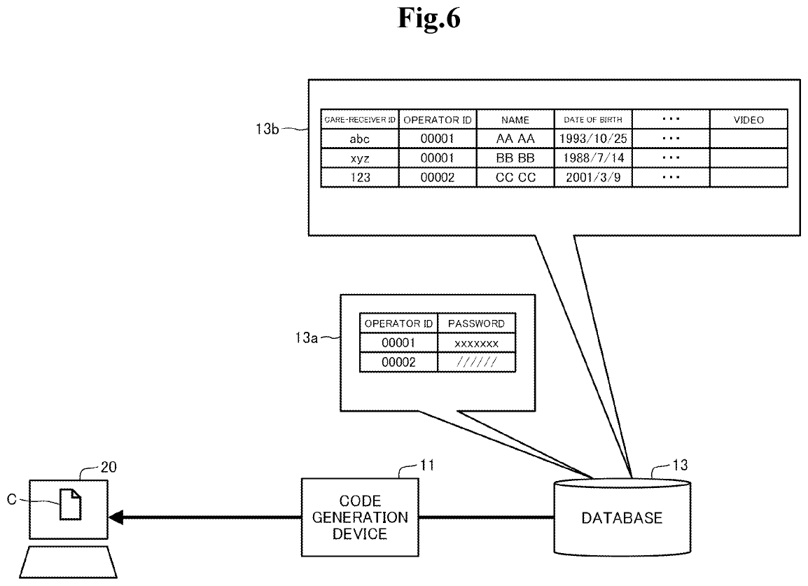

The operator operates the information processing device 20 to access the management system 10 over the network N, inputs login information that is operator information of the operator from the input device 22, and logs in to the management system (step S1 of FIGS. 4 and 5). The login information of the operator is authentication information including an operator ID and a password for authenticating the operator, and is stored in advance in a database 13 that is a storage device of the management system 10 as denoted by a reference numeral 13a in FIG. 6.

The operator who has logged in to the management system 10 from the information processing device 20 inputs care-receiver information of each care-receiver P with use of the input device 22 and registers it with the management system (step S2 of FIGS. 4 and 5). The care-receiver information of the care-receiver P includes, in addition to the care-receiver ID that is identification information for identifying the care-receiver P, the name and the date of birth of the care-receiver P, and also includes the operator ID of the operator who registers the care-receiver information. Then, as denoted by a reference numeral 13b in FIG. 6, the care-receiver information is stored in the database 13 that is a storage device of the management system 10.

Note that while the case of using the care-receiver ID as identification information of the care-receiver P is mainly shown below as an example, any information may be used as identification information if it is information unique to the care-receiver P. For example, as identification information of the care-receiver P, it is possible to use the name of the care-receiver P, or physical information indicating the physical characteristics extractable from a captured image of the care-receiver such as a face feature amount extractable from a face image of the care-receiver P. Note that the care-receiver information 13b is not limited to the information illustrated in FIG. 6, and may include any information such as a face image of the care-receiver P.

The management system 10 (third device) is configured of one or a plurality of information processing devices each having an arithmetic unit and a storage unit. As illustrated in FIG. 3, the management system 10 includes a code generation device 11 and an association device 12 constructed by execution of a program by the arithmetic unit. The management system 10 also includes the database 13 formed in the storage unit, and stores therein login information 13a for authenticating an operator and care-receiver information 13b for each care-receiver P. In the database 13, a video 50 related to the care-receiver P is to be stored, as described below.

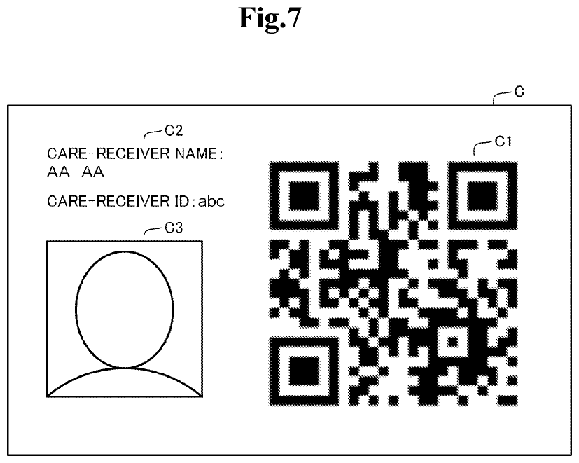

Then, the code generation device 11 issues a code C for each care-receiver P, based on the login information 13a and the care-receiver information 13b stored in the database 13 (step S3 of FIGS. 4 and 5). At this time, upon receiving a code issuance request along with designation of the care-receiver P from the operator via the information processing device 20 for example, the code generation unit 11 issues a code of the designated care-receiver P. Specifically, the code generation device 11 generates a QR code that is a matrix-type two-dimensional code including the care-receiver ID of the care-receiver P and the operator ID and the password of the operator associated with the care-receiver P in an encrypted manner. As an example, in the case of issuing a code of a care-receiver ID “abc” in the care-receiver information 13b illustrated in FIG. 6, the code generation device 11 generates a code C including information of the care-receiver ID “abc” of the care-receiver P, an operator ID “00001” and a password “******” of the operator ID “00001” who registered the care-receiver P. Then, the code generation device 11 outputs the generated code C from the output device 21 of the information processing device 20. At that time, as illustrated in FIG. 7, the code generation device 11 generates the code C including a code C1 itself, the care-receiver ID and the name C2 of the care-receiver P, and a face image C3 of the care-receiver P, and outputs the code C from the output device 21. It is assumed that the face image C3 of the care-receiver P is included in the care-receiver information 13b in advance and registered.

The code C generated as described above is output by being displayed on the display and is also output by being printed on a paper medium, by the output device 21 of the information processing device 20. The code C output by being printed is handed to the corresponding care-receiver P by the operator. For example, the operator refers to the name and the face image included in the code C, and hands the code C to the corresponding care-receiver P. At that time, as illustrated in FIG. 7, since the face image C3 of the care-receiver P is included in the code C, the operator can hand the code C by confirming the face image C3 of the code C and the face of the care-receiver P. Therefore, it is possible to reduce the possibility of erroneously imaging a different care-receiver P later. As described below, a video of the care-receiver P is captured with the code C held by the care-receiver P, which means that the code C is output in such a manner that the care-receiver ID and the operator ID and the password can be imaged.

The mobile terminal 30 (second device) is configured of an information processing device such as a smartphone having an arithmetic unit and a storage unit. As illustrated in FIG. 3, the mobile terminal 30 includes a reading device 31 and a video capture application 32 constructed by execution of a program by the arithmetic unit.

As described above, the operator hands the code C to the care-receiver P, and the care-receiver P performs motion for capturing a video while holding the code C such that the code C can be shown in the video (step S4 of FIGS. 4 and 5). The operator activates the video capture application 32 of the mobile terminal 30 to capture the video 50 of the care-receiver P holding the code C, and stores the video 50 in the storage unit of the mobile terminal 30 (steps S5 and S6 of FIGS. 4 and 5). Thereby, in the present embodiment, the video 50 is captured in which a scene of the care-receiver P and the code C are included in the same picture. Note that while the case of capturing a video by the mobile terminal 30 is described as an example in the present embodiment, an image to be captured may be any image including a still image. Moreover, while the case where the care-receiver P is shown in the video 50 is described as an example in the present embodiment, it is not limited to the case where the care-receiver P is shown. A scene related to the care-receiver P is shown is also acceptable. For example, the video 50 may be one captured by a camera mounted on a vehicle held by the care-receiver P. Moreover, in the video 50, it is not limited to the case where the code C is captured in the same picture. The video 50 and the code C may be captured as different images, and the images may be associated with each other. An example where the video 50 and the code C are captured as different images will be described later.

Then, during capturing of the video 50 including the code C by the mobile terminal 30 as described above, the reading device 31 detects the code C while capturing, and the mobile terminal 30 reads the content of the code C. That is, the reading device 31 reads, from the code C (code C1 itself) in the video 50, the login ID and the password that is login information of the operator and the care-receiver ID of the care-receiver P. Then, the reading device 31 accesses the management system 10, requests login with the readout login information, and makes an association request by requesting a search for the readout care-receiver ID (step S7 of FIGS. 4 and 5).

Then, in response to the association request from the mobile terminal 30, the association device 12 of the management system 10 performs a login process and searches for the care-receiver ID, based on the information registered in the database 13. Here, it is assumed that the care-receiver ID of the care-receiver ID “abc” and the login information of the operator ID “00001” who registered the care-receiver, as illustrated in FIG. 6, are transmitted from the mobile terminal 30 at the time of association request. In this case, the association device 12 checks the login information 13a and the care-receiver information 13b registered in the database 13, and when the login process, that is, authentication process, of the operator ID “00001” has succeeded and the care-receiver ID “abc” registered with the operator ID “00001” exists, the association device 12 sets to associate the video 50 with the care-receiver ID “abc”. Then, the association device 12 instructs the mobile terminal 30 to upload the video 50.

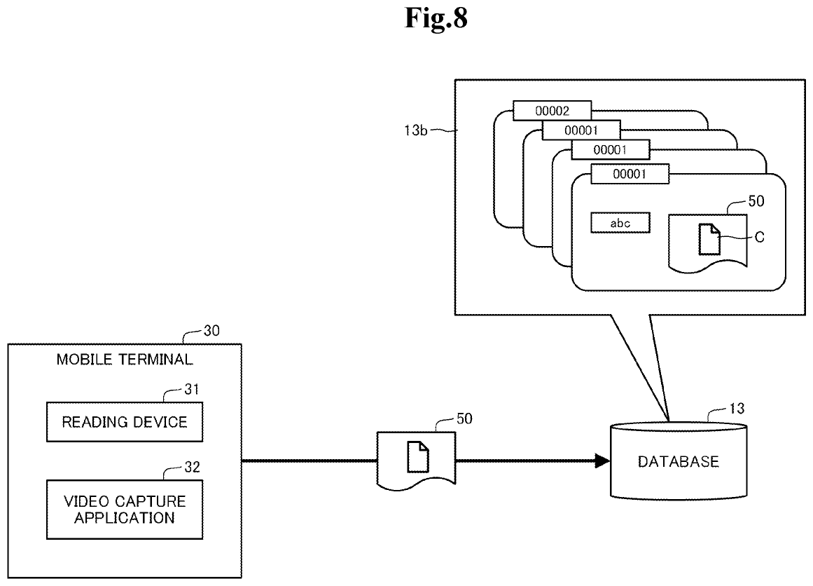

Then, after completion of capturing of the video 50, the video capture application 32 of the mobile terminal 30 uploads the video 50 to the management system 10 (step S8 of FIGS. 4 and 5). At that time, since the video 50 uploaded from the mobile terminal 30 is set to be associated with the care-receiver ID “abc” by the association device 12 of the management system 10 as described above, as illustrated in FIG. 8, it is stored in a state of being associated with the care-receiver ID “abc” in the database 13 of the management system 10. In this way, the video 50 is registered with the database 13 as a picture showing the scene related to the care-receiver of the care-receiver ID “abc”, and is managed in the management system 10 so as to be accessible by an authorized person.

After the upload of the video 50 is completed, the video capture application 32 of the mobile terminal 30 deletes the video 50 from the mobile terminal 30 from the viewpoint of protection of personal data (step S9 of FIG. 5). Note that when the association by the association device 12 described above has failed, that is, when the login process and searching for the care-receiver ID described above have failed, the mobile terminal 30 does not upload the video 50, and deletes the video 50 from the mobile terminal 30.

As described above, in the present embodiment, the code C including the login information of the operator and the care-receiver ID is issued, and the video 50 is captured while the care-receiver P holds the code C. Accordingly, it is possible to automatically extracts the login information of the operator and the care-receiver ID from the video 50. Therefore, in the mobile terminal 30 and the management system 10, a login process of the operator can be performed automatically, and the care-receiver P related to the video 50 can be specified automatically. Accordingly, it is possible to register the video 50 in association with the care-receiver P appropriately. As a result, the burden on the operator who operates to register the video 50, that is, the burden of the login process and the burden of a process of associating the video 50 with the care-receiver P can be reduced.

### <Modifications>

Next, description will be given on modifications of the configuration and the operation of the information processing system described above. In the above description, during capturing of the video 50, the code C in the video 50 is extracted at real time and an association request is made to the management system 10. However, an association request for the video 50 may be made after the video 50 has been captured. For example, as illustrated in steps S7 and S8 of FIG. 9, after the video 50 has been captured, the mobile terminal 30 may read the code C in the video 50 by the reading device 31, make an association request for the video 50 to the management system 10, and upload the video 50.

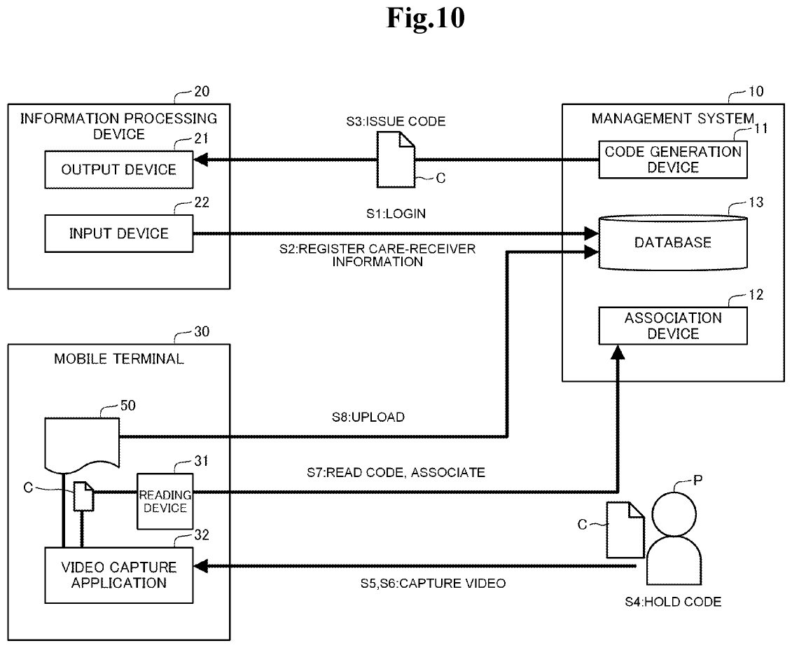

Further, in the above description, the video 50 is captured such that the code C is shown therein. However, the video 50 and the code C may be captured as different images. For example, as illustrated in FIG. 10, the mobile terminal 30 captures the video 50 and the code C separately by the video capture application 32. Then, the mobile terminal 30 extracts, by the reading device 31, the care-receiver ID and the login information in the same manner as described above from the image in which only the code C is captured, and makes an association request to the management system 10. Then, the management system 10 performs a login process and specifies the care-receiver P, and instructs the mobile terminal 30 to upload the video. In response to it, the mobile terminal 30 uploads the video 50 to the management system 10. Thereby, the management system 10 can receive the video 50 as being associated with the care-receiver ID included in the code C, and register the video 50 in association with the specified care-receiver ID. For example, the management system 10 may associate the code C and the video 50 transmitted within a certain period of time from the same mobile terminal 30, or associate them with each other in a different way.

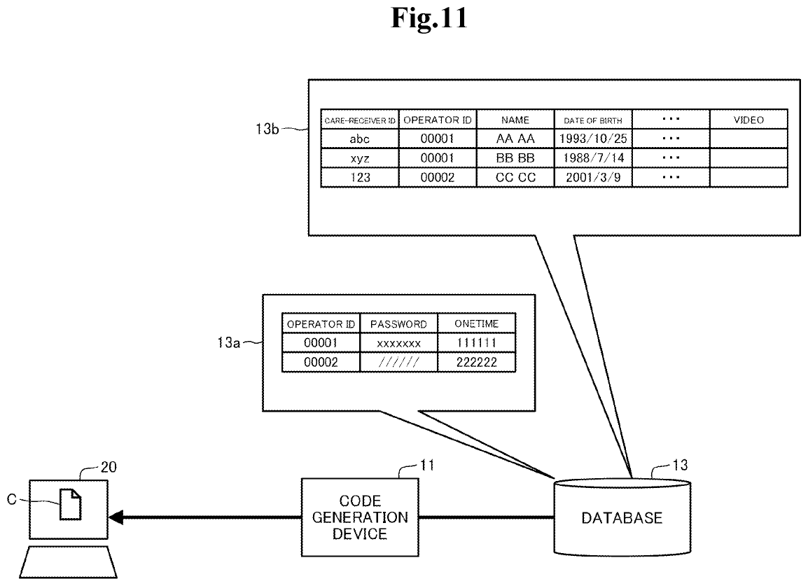

Moreover, as illustrated in FIG. 11, it is also possible to additionally register a onetime password for limiting the time and the number of times of login to the login information 13a (operator ID and password) of the operator registered in advance in the database 13, and generate the code C including the onetime password. Thereby, the management system 10 can limit the time and the number of times to the login request included in an association request that is made by reading the generated code C, whereby the security can be improved.

Further, in the example of FIG. 12, face information (face feature amount) of the care-receiver P is registered in advance in the care-receiver information 13b in the database 13. Further, the video capture application 32 of the mobile terminal 30 captures the video 50 so as to include the face image of the care-receiver P therein. The management system 10 further includes an authentication device 14, and the authentication device 14 performs face authentication to determine whether or not the face image of the care-receiver P shown in the video 50 and the face information registered in the care-receiver information 13b match. When the face authentication has succeeded, the management system 10 performs an association process of the video 50 based on the information included in the code C in the same manner as the above-described case. Note that authentication may be performed using physical information representing other physical characteristics of the care-receiver P, without limiting to the face of the care-receiver P shown in the video 50.

Note that while the case of including login information (operator ID and password) of the operator in the code C has been shown as an example in the above description, it is not necessary to include login information of the operator in the code C. This means that the code C may include only the identification information such as the care-receiver ID of the care-receiver P. Even in that case, the association device 12 of the management system 10 is able to associate the video 50 and the care-receiver P with each other and registers them with the database 13.

Moreover, while the code C including the care-receiver ID is issued in the above description, the code C may not be issued. In that case, as identification information of the care-receiver P, information of the care-receiver P shown in an image such as the video 50 is used. For example, in the care-receiver information 13b in the database 13, physical information representing physical characteristics such as the face and the flush amount of the care-receiver is registered in advance as identification information. Then, the mobile terminal 30 extracts physical information such as the face feature amount of the care-receiver P shown in the video 50 as identification information, and makes an association request to the management system 10. Thereby, the management system 10 specifies the care-receiver P matching the face feature amount of the care-receiver P shown in the video 50 from the care-receiver information 13b in the database 13, and associates the care-receiver P with the video 50 and registers them with the database 13.

### Second Exemplary Embodiment

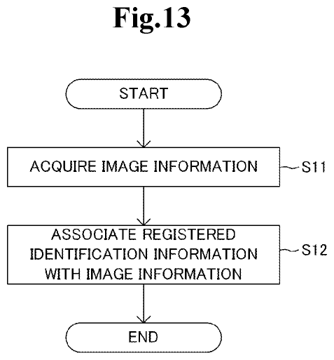

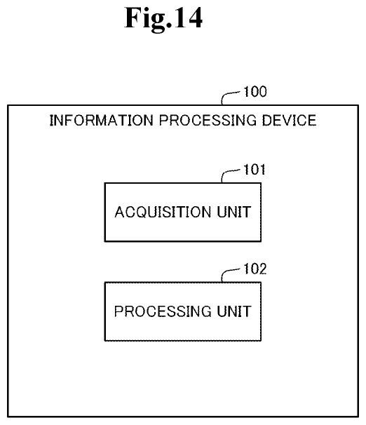

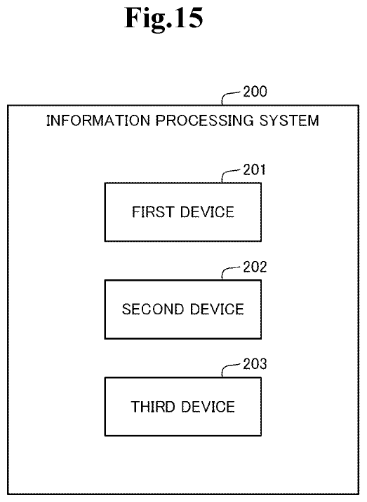

A second exemplary embodiment of the present invention will be described with reference to FIGS. 13 to 15. FIG. 13 is a flowchart illustrating an information processing method according to the present embodiment. FIG. 14 is a block diagram illustrating a configuration of an information processing device according to the present embodiment. FIG. 15 is a block diagram illustrating a configuration of an information processing system according to the present embodiment. The present embodiment shows the outline of the configuration and the operation of the information processing system described in the first exemplary embodiment.

As illustrated in FIG. 13, an information processing method of the present embodiment includes acquiring image information imaged in such a manner that identification information for identifying a user and a scene related to the user are associated with each other (step S11), and based on the image information, performing a process of associating the identification information registered in advance with the image information including the scene related to a user who is the same as the user identified by the identification information (step S12).

Then, the process by means of the information processing method is executed and implemented by an information processing device through execution of a program by the information processing device.

As illustrated in FIG. 14, the information processing method is also implemented by an information processing device 100 including an acquisition unit 101 that acquires image information imaged in such a manner that identification information for identifying a user and a scene related to the user are associated with each other, and a processing unit 102 that performs a process of associating the identification information registered in advance with the image information including the scene related to a user who is the same as the user identified by the identification information, based on the image information.

Moreover, as illustrated in FIG. 15, the information processing method is also implemented by an information processing system including:

a first device 201 that outputs identification information so as to be imagable, based on the identification information for identifying a user registered in advance;

a second device 202 that images image information in which the identification information output to be imagable and a scene related to the user identified by the identification information are associated with each other; and

a third device 203 that performs a process of associating the identification information registered in advance with the image information including the scene related to a user who is the same as the user identified by the identification information, based on the image information.

According to the invention as described above, identification information of a user can be automatically specified from image information imaged in such a manner that the identification information for identifying the user and a scene related to the user are associated with each other. Therefore, it is possible to automatically associate the specified user with image information including the scene related to the user. As a result, it is possible to reduce the burden on a worker who performs a work to associate an image with a user.

### <Supplementary Notes>

The whole or part of the exemplary embodiments disclosed above can be described as, but not limited to, the following supplementary notes. Hereinafter, outlines of the configurations of an information processing method. a program, an information processing device, and an information processing system, according to the present invention, will be described. However, the present invention is not limited to the configurations described below.

### (Supplementary Note 1)

An information processing method comprising:

acquiring image information imaged in such a manner that identification information for identifying a user and a scene related to the user are associated with each other; and

based on the image information, performing a process of associating the identification information registered in advance with the image information including the scene related to a user who is same as the user identified by the identification information.

### (Supplementary Note 2)

The information processing method according to supplementary note 1, wherein

the identification information and the scene are included in same image information, and the process of association is performed based on the image information.

### (Supplementary Note 3)

The information processing method according to supplementary note 1 or 2, further comprising:

outputting the identification information so as to be imagable, based on the identification information registered in advance;

imaging the image information in which the identification information output to be imagable and the scene related to the user identified by the identification information are associated with each other; and

performing the process of association based on the image information.

### (Supplementary Note 4)

The image processing method according to any of supplementary notes 1 to 3, wherein

the image information further includes authentication information of an operator who operates the process of association, and

the method further comprises:

authenticating the operator based on the authentication information of the operator included in the image information; and

performing the process of association based on the image information including the authentication information of the operator authenticated.

### (Supplementary Note 5)

The information processing method according to supplementary note 3 or 4, further comprising:

based on the identification information registered in advance and authentication information of an operator who operates the process of association registered in advance, outputting the identification information and the authentication information so as to be imagable;

imaging the image information in which the identification information and the authentication information, output to be imagable, and the scene related to the user identified by the identification information are associated with each other;

authenticating the operator based on the authentication information of the operator included in the image information; and

performing the process of association based on the image information including the authentication information of the operator authenticated.

### (Supplementary Note 6)

The image processing method according to any of supplementary notes 1 to 5, wherein

the identification information registered in advance is associated with physical information representing a physical characteristic of the user identified by the identification information, and

the method further comprises:

extracting the physical information of the user from the user shown in the image information; and

when the extracted physical information and the physical information associated with the identification information registered in advance match, performing the process of association based on the image information.

### (Supplementary Note 7)

The image processing method according to any of supplementary notes 1 to 6, wherein

the identification information registered in advance is associated with physical information representing a physical characteristic of the user identified by the identification information, and

the method further comprises:

outputting the identification information so as to be imagable based on the identification information registered in advance, and outputting by displaying the physical information associated with the identification information;

imaging the image information in which the identification information output to be imagable and the scene related to the user identified by the identification information are associated with each other; and

performing the process of association based on the image information.

### (Supplementary Note 8)

A program for causing an information processing apparatus to realize:

a process of acquiring image information imaged in such a manner that identification information for identifying a user and a scene related to the user are associated with each other, and based on the image information, performing a process of associating the identification information registered in advance with the image information including the scene related to a user who is same as the user identified by the identification information.

### (Supplementary Note 9)

An information processing device comprising:

an acquisition unit that acquires image information imaged in such a manner that identification information for identifying a user and a scene related to the user are associated with each other; and

a processing unit that performs, based on the image information, a process of associating the identification information registered in advance with the image information including the scene related to a user who is same as the user identified by the identification information.

### (Supplementary Note 9.1)

The information processing device according to supplementary note 9, wherein

the processing unit performs the process of association based on the image information in which the identification information and the scene are included.

### (Supplementary Note 10)

An information processing system comprising:

a first device that outputs identification information so as to be imagable, based on the identification information for identifying a user registered in advance;

a second device that images image information in which the identification information output to be imagable and a scene related to the user identified by the identification information are associated with each other; and

a third device that performs, based on the image information, a process of associating the identification information registered in advance with the image information including the scene related to a user who is same as the user identified by the identification information.

### (Supplementary Note 10.1)

The information processing system according to supplementary note 10, wherein

the second device images the image information in such a manner that the identification information and the scene are included in same image information.

### (Supplementary Note 10.2)

The information processing system according to supplementary note 10 or 10.1, wherein

the third device authenticates an operator based on authentication information of the operator who operates the process of association included in the image information, and performs the process of association based on the image information including the authentication information of the operator authenticated.

### (Supplementary Note 10.3)

The information processing system according to supplementary note 10 or 10.1, wherein

the first device outputs the identification information and the authentication information so as to be imagable, based on the identification information for identifying the user registered in advance and authentication information of an operator who operates the process of association registered in advance,

the second device images the image information in which the identification information and the authentication information, output to be imagable, and the scene related to the user identified by the identification information are associated with each other, and

the third device authenticates the operator based on the authentication information of the operator included in the image information, and performs the process of association based on the image information including the authentication information of the operator authenticated.

### (Supplementary Note 10.4)

The image processing system according to any of supplementary notes 10 to 10.3, wherein

the identification information registered in advance is associated with physical information representing a physical characteristic of the user identified by the identification information, and

the third device extracts the physical information of the user from the user shown in the image information, and when the extracted physical information and the physical information associated with the identification information registered in advance match, the third device performs the process of association based on the image information.

### (Supplementary Note 10.5)

The image processing system according to any of supplementary notes 10 to 10.4, wherein

the identification information registered in advance is associated with physical information representing a physical characteristic of the user identified by the identification information, and

the first device outputs the identification information so as to be imagable based on the identification information registered in advance, and displays and outputs the physical information associated with the identification information.

Note that the program described above can be supplied to a computer by being stored in a non-transitory computer readable medium of any type. Non-transitory computer readable media include tangible storage media of various types. Examples of non-transitory computer readable media include a magnetic recording medium (for example, flexible disk, magnetic tape, hard disk drive), a magneto-optical recording medium (for example, magneto-optical disk), a CD-ROM (Read Only Memory), a CD-R, a CD-R/W, a semiconductor memory (for example, mask ROM, PROM (Programmable ROM), and EPROM (Erasable PROM), a flash ROM, and a RAM (Random Access Memory). Note that the program described above may be supplied to a computer by being stored in a transitory computer readable medium of any type. Examples of transitory computer readable media include an electric signal, an optical signal, and an electromagnetic wave. A transitory computer readable medium can be supplied to a computer via a wired communication channel such as a wire and an optical fiber, or a wireless communication channel.

While the present invention has been described with reference to the exemplary embodiments described above, the present invention is not limited to the above-described embodiments. The form and details of the present invention can be changed within the scope of the present invention in various manners that can be understood by those skilled in the art.

The present invention is based upon and claims the benefit of priority from Japanese patent application No. 2019-006746, filed on Jan. 18, 2019, the disclosure of which is incorporated herein in its entirety by reference.

### REFERENCE SIGNS LIST

10 management system
11 code generation device
12 association device
13 database
13a login information
13b care-receiver information
14 authentication device
20 information processing device
21 output device
22 input device
30 mobile terminal
31 reading device
32 video capture application
50 video
C code
P care-receiver
100 information processing device
101 acquisition unit
102 processing unit
200 information processing system
201 first device
202 second device
203 third device

What is claimed is:

1. An information processing method comprising:
acquiring image information imaged in such a manner that identification information for identifying a user and a scene related to the user are associated with each other; and
based on the image information, performing a process of associating the identification information registered in advance with the image information including the scene related to a user who is same as the user identified by the identification information.

2. The information processing method according to claim 1, wherein
the identification information and the scene are included in same image information, and the process of association is performed based on the image information.

3. The information processing method according to claim 1, further comprising:
outputting the identification information so as to be imagable, based on the identification information registered in advance;
imaging the image information in which the identification information output to be imagable and the scene related to the user identified by the identification information are associated with each other; and
performing the process of association based on the image information.

4. The image processing method according to claim 1, wherein
the image information further includes authentication information of an operator who operates the process of association, and
the method further comprises:
authenticating the operator based on the authentication information of the operator included in the image information; and
performing the process of association based on the image information including the authentication information of the operator authenticated.

5. The information processing method according to claim 3, further comprising:
based on the identification information registered in advance and authentication information of an operator who operates the process of association registered in advance, outputting the identification information and the authentication information so as to be imagable;
imaging the image information in which the identification information and the authentication information, output to be imagable, and the scene related to the user identified by the identification information are associated with each other;
authenticating the operator based on the authentication information of the operator included in the image information; and
performing the process of association based on the image information including the authentication information of the operator authenticated.

6. The image processing method according to claim 1, wherein
the identification information registered in advance is associated with physical information representing a physical characteristic of the user identified by the identification information, and
the method further comprises:
extracting the physical information of the user from the user shown in the image information; and
when the extracted physical information and the physical information associated with the identification information registered in advance match, performing the process of association based on the image information.

7. The image processing method according to claim 1, wherein
the identification information registered in advance is associated with physical information representing a physical characteristic of the user identified by the identification information, and
the method further comprises:
outputting the identification information so as to be imagable based on the identification information registered in advance, and outputting by displaying the physical information associated with the identification information;
imaging the image information in which the identification information output to be imagable and the scene related to the user identified by the identification information are associated with each other; and
performing the process of association based on the image information.

8. (canceled)

9. An information processing device comprising:
an acquisition unit that acquires image information imaged in such a manner that identification information for identifying a user and a scene related to the user are associated with each other; and
a processing unit that performs, based on the image information, a process of associating the identification information registered in advance with the image information including the scene related to a user who is same as the user identified by the identification information.

10. The information processing device according to claim 9, wherein
the processing unit performs the process of association based on the image information in which the identification information and the scene are included.

11. An information processing system comprising:
a first device that outputs identification information so as to be imagable, based on the identification information for identifying a user registered in advance;
a second device that images image information in which the identification information output to be imagable and a scene related to the user identified by the identification information are associated with each other; and
a third device that performs, based on the image information a process of associating the identification information registered in advance with the image information including the scene related to a user who is same as the user identified by the identification information.

12. The information processing system according to claim 11, wherein
the second device images the image information in such a manner that the identification information and the scene are included in same image information.

13. The information processing system according to claim 11, wherein
the third device authenticates an operator based on authentication information of the operator who operates the process of association included in the image information, and performs the process of association based on the image information including the authentication information of the operator authenticated.

14. The information processing system according to claim 11, wherein
the first device outputs the identification information and the authentication information so as to be imagable, based on the identification information for identifying the user registered in advance and authentication information of an operator who operates the process of association registered in advance,
the second device images the image information in which the identification information and the authentication information, output to be imagable, and the scene related to the user identified by the identification information are associated with each other, and
the third device authenticates the operator based on the authentication information of the operator included in the image information, and performs the process of association based on the image information including the authentication information of the operator authenticated.

15. The image processing system according to claim 11, wherein
the identification information registered in advance is associated with physical information representing a physical characteristic of the user identified by the identification information, and
the third device extracts the physical information of the user from the user shown in the image information, and when the extracted physical information and the physical information associated with the identification information registered in advance match, the third device performs the process of association based on the image information.

16. The image processing system according to claim 11, wherein
the identification information registered in advance is associated with physical information representing a physical characteristic of the user identified by the identification information, and
the first device outputs the identification information so as to be imagable based on the identification information registered in advance, and outputs by displaying the physical information associated with the identification information.
